(19) United States
(12) Patent Application Publication
(10) Pub. No.: US 2006/0142271 A1
(43) Pub. Date: Jun. 29, 2006

(54) Novel lapacho compounds and methods of use thereof

(76) Inventors: Klaus Muller, Munster (DE); Andreas Sellmer, Lappersdorf (DE); June Salvesen, Bluffton, SC (US); Chiang Li, West Roxbury, MA (US); Youzhi Li, Westwood, MA (US); Xianggao Sun, Brookline, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/528,295

(22) PCT Filed: Sep. 17, 2003
(86) PCT No.: PCT/US03/29611

§ 371 Date: Oct. 25, 2005

Related U.S. Application Data
(60) Provisional application No. 60/411,478, filed on Sep. 17, 2002.
(60) Provisional application No. 60/441,338, filed on Jan. 21, 2003.

(51) Int. Cl. A61K 31/55

(52) U.S. Cl. 514/220

(57) ABSTRACT

The invention provides new synthetic lapacho derivatives as well as methods of use thereof. These compounds can be used in pharmaceutical compositions for the treatment or prevention of cell proliferation disorders. These compounds can also be used in the treatment or prevention of psoriasis or cancer or precancerous conditions.

### BACKGROUND OF THE INVENTION

Lapacho (“pau d'arco”, “ipê-roxo”, “taheebo”) is a commercial natural product obtained from the bark of Tabebuia trees, and in particular from T. impetiginosa (Martius ex DC.) Standley (3inoniaceae), which are found in the rainforests throughout Central and South America. Lapacho has been used as a folk medicine for many years, in particular for the treatment of cancer (Hartwell, J. L., Lloydia, 31, 71-170, 1968) and disorders of the immune system, including psoriasis (Jones, I, Pau D'arco: Immune Power from the Rain Forest; Healing Arts Press; Rochester, Vt., 1995).

The occurrence of naphthoquinones in various members of the genus Tabebuia has been widely reported (Burnett, A. R., et. al., J. Chem. Soc., C, 2100-2104, 1967; Rao, M. M, et. al, J. Nat. Prod., 45, 600-604, 1982; Girard, M., et. al., J. Nat. Prod., 51, 1023-1024, 1988; and Diaz, F., et. al., J. Nat. Prod., 59, 423424, 1996). The best known of these compounds are lapachol, alpha-lapachone (α-lapachone) and beta-lapachone (β-lapachone), which have the following chemical structures:

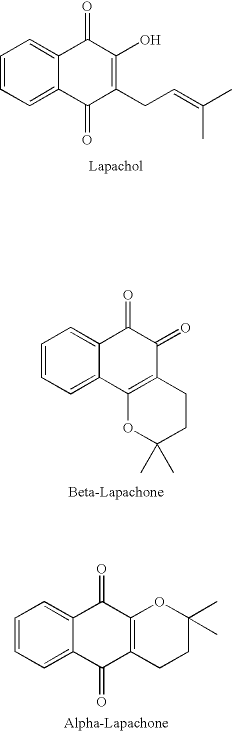

Although all three of these compounds have been reported to have antiproliferative activity, β-lapachone, in particular, has demonstrated significant antineoplastic activity against a wide spectrum of human cancer cell lines at concentrations typically in the range of 1-10 μM (IC₅₀). For example, the cytotoxicity of β-lapachone has been demonstrated in transformed cell lines derived from patients with promyelocytic leukemia (Planchon et al., Cancer Res., 55 (1996) 3706), prostate ( Li, C J., et al., Cancer Res., 55 (1995) 3712), malignant glioma (Weller, M. et al., Int. J. Cancer, 73 (1997) 707), hepatoma (Lai, C. C., et al., Histol Histopathol, 13 (1998) 8), colon (Huang, L., et al., Mol Med, 5, (1999) 711), breast (Wuertzberger, S. M., et al., Cancer Res., 58 (1998) 1876), ovarian (Li, C. J. et al., Proc. Natl. Acad. Sci. USA, 96(23) (1999) 13369-74), pancreatic (Li, Y., et al., Mol Med, 6 (2000) 1008; Li, Y. Z., Mol Med, 5 (1999) 232), and multiple myeloma cell lines, including drug-resistant lines (Li, Y., Mol Med, 6 (2000) 1008). No cytotoxic effects were observed on normal fresh or proliferating human PBMC (Li, Y., Mol Med, 6 (2000) 1008).

Other lapacho-derived compounds have been shown to have antiproliferative activity. Eight compounds, representing the most common constituents of the inner bark of T. impetiginosa and including lapachol, α-lapachone and β-lapachone, were evaluated for antiproliferative and cytotoxic activity in the nontransformed human keratinocyte cell line HaCaT, a model for the highly proliferative epidermis characteristic of psoriasis (Müller, K., et al., J. Nat. Prod. 62 (1999) 1134-1136). While lapachol and α-lapachone were relatively inactive in this model, β-lapachone and several naphtho[2,3-b]furan diones displayed inhibition of keratinocyte growth comparable to the antipsoriatic drug anthralin. These findings encourage the design and synthesis of new lapacho compounds and their evaluation for antiproliferative activity in a variety of biological systems.

### SUMMARY OF THE INVENTION

The present invention provides new synthetic lapacho derivatives of Formula I: 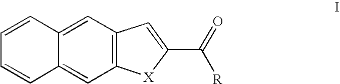
wherein X is O or S; and R is straight-chained or branched C₁-C₆ alkyl, aryl, substituted aryl (substituted, for example, with: hydroxyl, alkoxy, C₁-C₆ alkyl, nitro, halogen carboxyl, carboxyalkyl), or straight-chained or branched alkylaryl, or a pharmaceutically acceptable salt thereof; wherein 1) R is not methyl; 2) where X is O, R is not bromomethyl, unsubstituted phenyl, or phenyl substituted at the 4-position with methyl, chloro, ethenyl, or 2′-chloroethyl; 3) where X is S, R is not 2-carboxyphenyl. In preferred embodiments, R is an aryl group; in more preferred embodiments, the aryl group is a phenyl group substituted with one or two hydroxyl or alkyloxy groups (preferably alkoxy groups); in still more preferred embodiments, the phenyl group is substituted with one or two methoxy groups; more preferably, the phenyl group is a 3,4-dimethoxyphenyl group.

The present invention also provides new synthetic lapacho derivatives of Formula II: 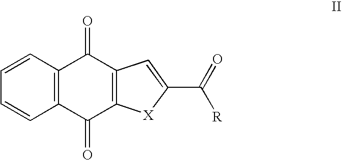
wherein X is O or S; and R is straight-chained or branched C₁-C₆ alkyl, aryl, substituted aryl (substituted, for example, with: hydroxyl, alkoxy, C₁-C₆ alkyl, nitro, halogen carboxyl, carboxyalkyl), or straight-chained or branched alkylaryl, or a pharmaceutically acceptable salt thereof; wherein R is not methyl. In preferred embodiments, R is an aryl group; in more preferred embodiments, the aryl group is a phenyl group substituted with one or two hydroxyl or alkyloxy groups; in still more preferred embodiments, the phenyl group is substituted with one or two methoxy groups; more preferably, the phenyl group is a 3,4-dimethoxyphenyl group.

The present invention also concerns new synthetic lapacho analogs of Formula III: 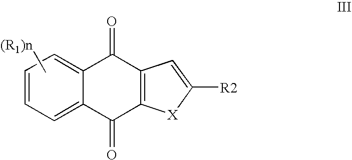
wherein X is O or S; R₁ is independently at each incidence hydroxyl, alkoxyl, C₁-C₆ alkyl, nitro, halogen, carboxyl or carboxyalkyl; R₂ is hydrogen or —C(O)—R₃, R₃ is straight-chained or branched C₁-C₆ alkyl, aryl, substituted aryl (substituted, for example, with: hydroxyl, alkoxy, C₁-C₆ alkyl, nitro, halogen, carboxyl, carboxyalkyl), or straight-chained or branched alkylaryl; and n is 0, 1 or 2; or a pharmaceutically acceptable salt thereof; wherein 1) where X is O, R₂ is not H; 2) where X is O, and R₂ is —C(O)—R₃, and R₃ is methyl, then R₁ is not hydroxyl or methoxy; and 3) where X is S and R₂ is H, then n is 1 and R₁ is selected from —OH and —OC(O)-alkyl(C₁-C₆); and 4) where X is S and R₂ is —C(O)—R₃, and R₃ is methyl, then R₁ does not represent a 7-acetyl group.

Preferred compounds of Formula I are those in which X is S and R is aryl or substituted aryl.

Preferred compounds of Formula II are those in which X is O or S and R is alkyl, aryl or mono- or di-substituted aryl.

Preferred compounds of Formula III are those in which X is S, R₁ is hydroxyl or —OC(O)-alkyl(C₁-C₆), R₂ is hydrogen, and n is 1; still more preferably, R₁ is 5-hydroxyl or 5—OC(O)-methyl.

The present invention also provides pharmaceutical formulations comprising a compound of Formula I, II or III in combination with at least one pharmaceutically acceptable excipient or carrier.

The present invention also provides a method for the treatment of cell proliferative disorders in mammals comprising administering to a mammal in need of such treatment an effective amount of a compound of Formula I, II or III. The invention further provides the use of a compound of Formula I, II or III for the preparation of a medicament useful for the treatment of a cell proliferative disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides new synthetic lapacho derivatives of Formula I: 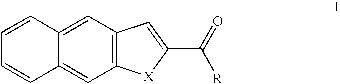
wherein X is O or S; and R is straight-chained or branched C₁-C₆ alkyl, aryl, substituted aryl (substituted, for example, with from one to four of the following moieties: hydroxyl, alkoxy, C₁-C₆ alkyl, nitro, carboxyl, carboxyalkyl), or straight-chained or branched alkylaryl, or a pharmaceutically acceptable salt thereof; wherein 1) R is not methyl; 2) where X is O, R is not bromomethyl, unsubstituted phenyl, or phenyl substituted at the 4-position with methyl, chloro, ethenyl, or 2′-chloroethyl; 3) where X is S, R is not 2-carboxyphenyl. In preferred embodiments, R is an aryl group; in more preferred embodiments, the aryl group is a phenyl group substituted with one or two hydroxyl or alkoxy groups (preferably alkoxy groups); in still more preferred embodiments, the phenyl group is substituted with one or two methoxy groups; more preferably, the phenyl group is a 3,4-dimethoxyphenyl group.

Preferred compounds of Formula I are those in which X is S and R is aryl or substituted aryl. For example, aryl can have 1, 2, 3, or 4 substituents, which can be the same or different. Preferably, the aryl group has 1, 2, 3, or 4 substituents independently selected from hydroxyl, alkoxy, alkyl, nitro, halogen, carboxyl or carboxyalkyl.

The present invention also provides new synthetic lapacho derivatives of Formula II: 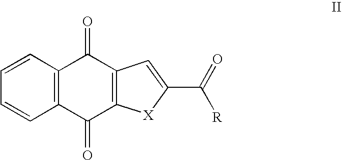
wherein X is O or S; and R is straight-chained or branched C₁-C₆ alkyl, aryl, substituted aryl (substituted, for example, with from one to four of the following moieties: hydroxyl, alkoxy, C₁-C₆ alkyl, nitro, carboxyl, carboxyalkyl), or straight-chained or branched allylaryl, or a pharmaceutically acceptable salt thereof; wherein R is not methyl. In preferred embodiments, R is an aryl group; in more preferred embodiments, the aryl group is a phenyl group substituted with one or two hydroxyl or alkyloxy groups; in still more preferred embodiments, the phenyl group is substituted with one or two methoxy groups; more preferably, the phenyl group is a 3,4-dimethoxyphenyl group.

Preferred compounds of Formula II are those in which X is O or S and R is alkyl, aryl or mono- or di-substituted aryl. For example, aryl can have 1, 2, 3, or 4 substituents, which can be the same or different Preferably, the aryl group has 1, 2, 3, or 4 substituents independently selected from hydroxyl, alkoxy, alkyl, nitro, halogen, carboxyl or carboxyalkyl.

The present invention also concerns new synthetic lapacho analogs of Formula III: 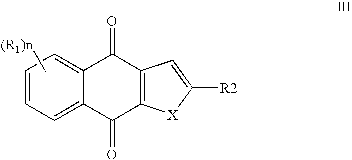
wherein X is O or S; R₁ is independently at each incidence hydroxyl, alkoxyl, C₁-C₆ alkyl, nitro, halogen, carboxyl or carboxyalkyl; R₂ is hydrogen or —C(O)—R₃, R₃ is straight-chain or branched C₁-C₆ alkyl, aryl, substituted aryl (substituted, for example, with from one to four of the following moieties: hydroxyl, alkoxy, C₁-C₆ alkyl, nitro, carboxyl, carboxyalkyl), or straight-chained or branched alkylaryl; and n is 0, 1 or 2; or a pharmaceutically acceptable salt thereof; wherein 1) where X is O, R₂ is not H; 2) where X is O, and R₂ is —C(O)—R₃, and R₃ is methyl, then R₁ is not hydroxyl or methoxy; and 3) where X is S and R₂ is H, then n is 1 and R₁ is selected from —OH and —OC(O)-alkyl(C₁-C₆); and 4) where X is S and R₂ is —C(O)—R₃, and R₃ is methyl, then R₁ does not represent a 7-acetyl group.

Preferred compounds of Formula III are those in which X is O, R₁ is hydroxyl or —OC(O)-alkyl(C₁-C₆), R₂ is hydrogen, and n is 1; still more preferably, R₁ is 5-hydroxyl or 5—OC(O)-methyl.

Other preferred compounds of Formula III are those in which X is O or S, R₂ is hydrogen or —C(O)—R₃. In embodiments where R₂ is —C(O)—R₃, R₃ is preferably an aryl group. For example, aryl can have 1, 2, 3, or 4 substituents, which can be the same or different. Preferably, the aryl group has 1, 2, 3, or 4 substituents independently selected from hydroxyl, alkoxy, alkyl, nitro, halogen, carboxyl or carboxyalkyl.

In more preferred embodiments, the aryl group is a substituted or unsubstituted phenyl group. In certain preferred compounds of Formula III where R₂ is —C(O)—R₃, R₃ is a phenyl group substituted with one or two hydroxyl or alkyloxy groups; in still more preferred embodiments, the phenyl group is substituted with one or two methoxy groups; more preferably, the phenyl group is a 3,4-dimethoxyphenyl group, wherein when n is 0, R₃ is not methyl.

Certain preferred compounds of the invention are shown in Tables 1-4.

The term “alkyl” refers to radicals containing carbon and hydrogen, without unsaturation. Alkyl radicals can be straight or branched. Exemplary alkyl radicals include, without limitation, methyl, ethyl, propyl, isopropyl, hexyl, t-butyl, sec-butyl and the like. A C₁-C₆ alkyl group is an alkyl group having from one to six carbon atoms in the straight or branched alkyl backbone. Alkyl groups optionally can be substituted with one or more moieties such as hydroxyl group, carboxylate, oxo, halogen, thiol, cyano, nitro, amino, acylamino, C₁-C₆ alkylthio, arylthio, C₁-C₆ alkyl, C₁-C₆ alkoxy, aryloxy, alkylcarbonyloxy, arylcarbonyloxy, C₃-C₆ cycloalkyl, C₃-C₆ cycloalkyloxy, C₂-C₆ alkenyl, C₂-C₆ alkynyl, aryl, aminocarbonyl, C₁- C₆ alkylcarbonyl, C₃-C₆ cycloalkylcarbonyl, heterocyclylcarbonyl, arylcarbonyl, aryloxycarbonyl, C₁-C₆ alkoxycarbonyl, C₃-C₆ cycloalkyloxycarbonyl, heterocyclyloxycarbonyl, C₁-C₆ alkylsulfonyl, arylsulfonyl, a heterocyclyl group, and the like.

Alkyl radicals can be cyclic. A “cycloalkyl” group refers to a cyclic alkyl group which has a ring having from three to six carbon atoms in the ring portion. A cycloalkyl group may be substituted with one or moieties as described for alkyl groups.

As used herein, the term “aryl” refers to an aromatic carbocyclic or heteroaromatic moiety, having one, two, or three rings. An aryl group may be carbocyclic or may optionally contain from 1-4 heteroatoms (such as nitrogen, sulfur, or oxygen) in the aromatic ring. Exemplary aryl groups include, without limitation, phenyl, naphthyl, pyridyl, pyrimidyl, triazinyl, quinazolinyl, thiazolyl, benzothiophenyl, furanyl, imidazolyl, thiophenyl and the like. An aryl group optionally can be substituted with one or more substituents such as hydroxyl group, halogen, thiol, cyano, nitro, amino, acylamino, C₁-C₆ alkylthio, arylthio, C₁-C₆ alkyl, C₁-C₆ alkoxy, aryloxy, alkylcarbonyloxy, arylcarbonyloxy, C₃-C₆ cycloalkyl, C₃-C₆ cycloalkyloxy, C₂-C₆ alkenyl, C₂-C₆ alkynyl, aryl, carboxylate, aminocarbonyl, C₁-C₆ alkylcarbonyl, C₃-C₆ cycloalkylcarbonyl, heterocyclylcarbonyl, arylcarbonyl, aryloxycarbonyl, C₁-C₆ alkoxycarbonyl, C₃-C₆ cycloalkyloxycarbonyl, heterocyclyloxycarbonyl, aryloxycarbonyl, C₁-C₆ alkoxycarbonyl, C₁-C₆ alkylsulfonyl, arylsulfonyl, a heterocyclyl group, and the like.

The term “heterocyclyl” or “heterocycle” refers to a stable non-aromatic 3-7 membered monocyclic heterocyclic ring or 7-11 membered bicyclic heterocyclic ring which is either saturated or unsaturated, and may be fused, spiro or bridged to form additional rings. Each heterocycle consists of one or more carbon atoms and from one to four heteroatoms selected from the group consisting of nitrogen, oxygen and sulfur. A heterocyclyl radical may be attached at any endocyclic atom which results in the creation of a stable structure. Preferred heterocycles include 3-7 membered monocyclic heterocycles (more preferably 5-7-membered monocyclic heterocycles) such as (without limitation) piperidinyl, pyranyl, piperazinyl, morpholinyl, thiamorpholinyl, and tetrahydrofuranyl.

In general, structures depicted herein are meant to include all stereochemical forms of the structure; i.e., the R and S configurations for each asymmetric center, unless a particular stereochemistry is specifically indicated. Therefore, single stereochemical isomers (i.e., substantially pure enantiomers and diasteromers) as well as enantiomeric and diastereomeric mixtures, such as racemic mixtures, of the present compounds are within the scope of the invention. Furthermore, all geometric isomers, such as E- and Z-configurations at a double bond, are within the scope of the invention unless otherwise stated. Certain compounds of this invention may exist in tautomeric forms. All such tautomeric forms of the compounds are considered to be within the scope of this invention unless otherwise stated.

The present invention also provides pharmaceutical formulations comprising a compound of Formula I, II or III in combination with at least one pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in “Remington: The Science and Practice of Pharmacy, Twentieth Edition,” Lippincott Williams & Wilkins, Philadelphia, Pa., which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A compound of Formula I, II, or III is administered in a suitable dosage form prepared by combining a therapeutically effective amount (e.g., an efficacious level sufficient to achieve the desired therapeutic effect through inhibition of tumor growth, killing of tumor cells, etc.) of a compound of Formula I, II, or III (as an active ingredient) with standard pharmaceutical carriers or diluents according to conventional procedures (i.e., by producing a pharmaceutical composition of the invention). These procedures may involve mixing, granulating, and compressing or dissolving the ingredients as appropriate to attain the desired preparation.

Preferred pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, ethylcellulose, hydroxypropylmethylcellulose, methylmethacrylate or the like. Other fillers, excipients, flavorants, and other additives such as are known in the art may also be included in a pharmaceutical composition according to this invention.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers comprising excipients and/or auxiliaries which facilitate processing of the active compounds into preparations that can be used pharmaceutically. Of course, the appropriate formulation is dependent upon the route of administration chosen.

A compound or pharmaceutical composition of the invention can be administered to a subject in many of the well-known methods currently used for chemotherapeutic treatment For example, for treatment of cancers, a compound of the invention may be injected directly into tumors, injected into the blood stream or body cavities or taken orally or applied through the skin with patches. For treatment of psoriatic conditions, systemic administration (e.g., oral administration), or topical administration to affected areas of the skin, are preferred routes of administration. The dose chosen should be sufficient to constitute effective treatment but not so high as to cause unacceptable side effects. The state of the disease condition (e.g., cancer, psoriasis, and the like) and the health of the patient should preferably be closely monitored during and for a reasonable period after treatment.

The present invention also provides a method for the treatment of cell proliferative disorders in mammals comprising administering to a mammal an effective amount of a compound of Formula I, II or III. The mammal is preferably a mammal in need of such treatment. The mammal can be e.g., any mammal, e.g., a human, a primate, mouse, rat, dog, cat, cow, horse, pig. In a preferred embodiment, the mammal is a human. The invention further provides the use of a compound of Formula I, II or III for the preparation of a medicament useful for the treatment of a cell proliferative disorder. The compounds of the invention are preferably administered in the form of pharmaceutical compositions, e.g., as described herein.

As used herein, the term “cell proliferative disorder” refers to conditions in which the unregulated and/or abnormal growth of cells can lead to the development of an unwanted condition or disease, which can be cancerous or non-cancerous, for example a psoriatic condition. As used herein, the term “psoriatic condition” refers to disorders involving keratinocyte hyperproliferation, inflammatory cell infiltration, and cytokine alteration.

In addition to psoriatic conditions, the types of proliferative diseases which may be treated using the compositions of the present invention are epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses and the like.

The invention will be further described in the following examples, which do not limit the scope of the invention described in the claims.

### EXAMPLES

### Example 1.

### Synthesis of Synthetic Lapcho Analogs

Compounds of the invention can be prepared in a variety of ways, some of which are known in the art. In general, the compounds of the present invention can be prepared from commercially available starting materials, compounds known in the literature, or from readily-prepared intermediates, by employing standard synthetic methods and procedures known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}.; John Wiley & Sons: New York, 1999 are useful and recognized reference textbooks of organic synthesis known to those in the art. The following descriptions of synthetic methods are designed to illustrate, but not limit, general procedures for the preparation of compounds of the invention.

Melting points were determined with a Reichert Thermovar melting point apparatus and are uncorrected. Chromatography refers to column chromatography on silica gel (E. Merck, 70-230 mesh) using CH₂Cl₂ as eluant, unless otherwise stated. ¹H NMR spectra were recorded with a Varian EM 390 (90 MHz) or a Bruker Spectrospin WM 250 spectrometer (250 MHz), using tetramethylsilane as an internal standard. Fourier-transform IR spectra (KBr) were recorded on a Nicolet 510M FTIR spectrometer. UV spectra were recorded on a Kontron 810 spectrometer. Mass spectra (EI) were obtained on a Varian MAT 112S spectrometer (70 eV). Elemental Analysis were within ±0.4% of calculated values.

### Example 2.

### Analogs of Formulae I and II

One process that can be used for the preparation of the most preferred compounds of Formula I and II is shown in Scheme 1. Introduction of the 2-acyl functionality onto the naphtho[2,3-b]thiophene and naphtho[2,3-b]furan nuclei was achieved by metalation with sec-butyllithium in the presence of tetramethylethylenediamine, where substitution occurs exclusively in the 2-position. The reaction of naphtho[2,3-b]thiophene- and naphtho[2,3-b]furan-2-yl-lithium with the appropriate aldehydes gave the secondary alcohols 62-66 and 73-75, respectively. The desired acyl group was obtained by oxidation of the alcohol group with activated manganese(IV) oxide in methylene chloride. Oxidation of the 2-acyl analogues with chromium trioxide in glacial acetic acid provided the corresponding naphtho[2,3-b]thiophene- and naphtho[2,3-b]furan4,9-diones 62b-66b and 6, 74b, 75b, respectively. The phenolic analogues 62c and 65c were obtained by ether cleavage of the corresponding methyl ethers 62b and 65b with boron tribromide in methylene chloride. 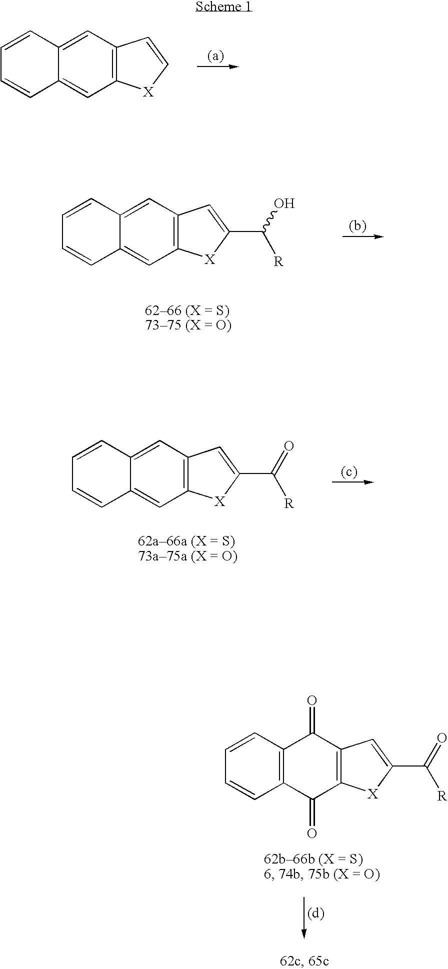
Reagents: (a) sec-BuLi, tetramethylethylenediamine, ether, −78°; (b) MnO₂, CH₂Cl₂; (c) CrO₃, HOAc; (d) BBr₃, CH₂Cl₂. R and X are defined in Tables 1 and 2.

### Compounds of Scheme 1

General Procedure for the Preparation of (R,S)-Naphtho[2,3-b]thiophen-2-yl-alkanols. (R,S)-(4-Methoxyphenyl)-naphtho[2,3-b]thiophen-2-yl-methanol (62). To a solution of naphtho[2,3-b]thiophene (0.92 g, 4.99 mmol) in absolute Et₂O (80 mL) and tetramethylethylenediamine (0.08 mL, 0.8 mmol) was added sec-butyllithium (4.40 mL of a 1.3 M solution in hexane, 5.72 mmol) at −78° C. under N₂. Then the solution was stirred at −78° C. for 1 h. Dry 4-methoxybenzaldehyde (0.73 mL, 6.0 mmol), freshly distilled, was added at −78° C., and the solution was allowed to warm to room temperature within 12 h. Then it was treated with a solution of half-saturated NH₄Cl (400 mL), the organic layer was washed with water (400 mL), dried over Na₂SO₄, and evaporated. The residue was purified by chromatography and recrystallized from CH₂Cl₂/hexane to afford white crystals: 72% yield; mp 164-165° C.; FTIR 3377 (OH), 1611 cm⁻¹; ¹H NMR (CDCl₃) δ 8.25-6.90 (m, 11H), 6.90 (d, 1H, ³J=4.01 Hz), 3.82 (s, 3H), 2.47 (d, 1H, ³J=4.01 Hz, exchangeable); MS, m/z 320 (66, M³⁰ ), 135 (100). Anal. (C₂₀H₁₆O₂S) C, H.

(R,S)-Naphtho[2,3-b]thiophen-2-yl-ethanol (63) was obtained from naphtho[2,3-b]thiophene (1.00 g, 5.43 mmol) and acetaldehyde (0.37 mL, 6.52 mmol) as described for 62 to afford white needles: 44% yield; mp 182-184° C.; FTIR 3319 (OH) cm¹; ¹H NMR (CDCl₃) δ8.23-7.43 (m, 6H), 7.25 (s, 1H), 5.21 (qu, 1H, ³J=6.42 Hz), 1.82 (d, 1H, ³J=6.42 Hz, exchangeable), 1.69 (s, 3H). Anal. (C₁₄H₁₂OS) C, H.

(R,S)-Naphtho[2,3-b]thiophen-2-yl-phenylmethanol (64) was obtained from naphtho[2,3-b]thiophene (0.75 g, 4.07 mmol) and benzaldehyde (0.49 mL, 4.88 mmol) as described for 62 to afford white needles: 67% yield; mp 142-145° C.; FTIR 3319 (OH) cm⁻¹; ¹H NMR (CDCl₃) δ8.56-7.16 (m, 12H), 6.13 (d, 1H, ³J=3.59 Hz), 2.58 (d, 1H, ³J=3.59 Hz, exchangeable). Anal. (C₁₉H₁₄OS) C, H.

(R,S)-(3,4-Dimethoxyphenyl)-naphtho[2,3-b]thiophen-2-yl-methanol (65) was obtained from naphtho[2,3-b]thiophene (1.00 g, 5.43 mmol) and 3,4-dimethoxybenzaldehyde (1.08 g, 6.51 mmol) as described for 62 to afford white crystals: 65% yield; mp 175-176° C.; FTIR 3481 (OH), 1594 cm⁻¹; ¹H NMR (CDCl₃) δ8.43-6.93 (m, 10H), 6.40 (d, 1H,³J=3.00 Hz, exchangeable), 6.00 (d, 1H, ³J=3.00 Hz), 3.81 (s, 3H), 3.80 (s, 3H). Anal. (C₂₁H₁₈O₃S) C, H.

(R,S)-Naphtho[2,3b]thiophen-2-yl-(4-nitrophenyl)methanol (66) was obtained from naphtho[2,3-b]thiophene (1.00 g, 5.43 mmol) and 4nitrobenzaldehyde (1.07 g, 7.06 mmol) as described for 62 to afford light-yellow crystals: 55% yield; mp 239-240° C.; FTIR 3548 (OH), 1596 cm⁻¹; ¹H NMR (DMSO-d₆) δ8.67-7.43 (m, 11H), 6.84 (d, 1I, ³J=4.31 Hz, exchangeable), 6.27 (d, 1H, ³J=4.31 Hz). Anal. (C₁₉H₁₃NO₃S) C, H.

General Procedure for the Preparation of Naphtho[2,3-b]thiophen-2-yl-akanones. (4-Methoxyphenyl)-naphtho[2,3-b]thiophen-2-yl-methanone (62a). To a solution of 62 (1.00 g, 3.12 mmol) in CH₂Cl₂ (100 mL) was added activated MnO₂ (2.61 g, 30 mmol), and the mixture was stirred for 1.5 h, until the oxidation was completed (TLC control). The suspension was filtered, and the residue was washed with CH₂Cl₂ (3×200 mL). The solution was treated with hexane (500 mL), then concentrated, and the product was crystallized at −18° C. to afford lemon crystals: 81% yield; mp 211-212° C.; FTIR 1622 (CO), 1603 cm⁻¹; ¹H NMR (CDCl₃) δ8.43-7.01 (m, 11H), 3.92 (s, 3H); MS nmz 318 (100, M³⁰ ). Anal. (C₂₀H₁₄O₂S) C, R

Naphtho[2,3-b]thiophen-2-yl-ethanone (63a) was obtained from 63 (0.35 g, 1.53 mmol) as described for 62a, but it was stirred for 24 h to afford greenish-yellow needles: 84% yield; mp 224-225° C.; FTIR 1663 (CO) cm⁻¹; ¹H NMR (DMSO-d₆) 68.67-7.51 (m, 7H), 2.71 (s, 3H). Anal. (C₁₄H₁₀OS) C, H.

Naphtho[2,3-b]thiophen-2-yl-phenylmethanone (64a) was obtained from 64 (0.35 g, 1.21 mmol) as described for 62a to afford yellow crystals: 96% yield; mp 164-165° C.; FTIR 1630 (CO), 1595 cm⁻¹; ¹H NMR (CDCl₃) δ8.42-7.45 (m, 12H). Anal. (C₁₉H₁₂OS) C, H.

(3,4-Dimethoxyphenyl)-naphtho[2,3-b]thiophen-2-yl-methanone (65a) was obtained from 65 (0.91 g, 2.60 mmol) as described for 62a to afford lemon crystals: 87% yield; mp 175-176° C.; FTIR 1630 (CO), 1596 cm⁻¹; ¹H NMR (CDCl₃) 88.44-6.97 (m, 10H), 4.00 (s, 3H), 3.98 (s, 3H). Anal. (C₂₁H₁₆O₃S) C, H.

Naphtho[2,3-b]thiophen-2-yl-(4-nitrophenyl)methanone (66a) was obtained from 66 (0.50 g, 1.49 mmol) as described for 62a to afford orange crystals: 97% yield; mp 251-252° C.; FTIR 1628 (CO), 1600 cm⁻¹; ¹H NMR (CDCl₃) δ8.78-7.48 (m, 11H). Anal. (C₁₉H₁₁NO₃S) C, H.

General Procedure for the Oxidation of Naphtho[2,3-b]thiophenes to Naphtho[2,3-b]thiophene-4,9-diones. 2-(4-Methoxybenzoyl)-naphtho[2,3-b]thiophene-4,9-dione (62b). To a solution of 62a (0.75 g, 2.35 mmol) in glacial acetic acid (50 mL) was added with stirring at room temperature, dropwise over 1 h, a solution of CrO₃ (0.66 g, 6.6 mmol) in glacial acetic acid (10 mL) and water (10 mL). The solution was stirred for an additional 30 min, then water (250 mL) was added, the product was filtered by suction, and purified by chromatography. The combined fractions were treated with hexane, then concentrated, and the product was crystallized at −18° C. to afford lemon needles: 86% yield; mp 165-166° C.; FTIR 1667 (CO), 1600 cm⁻¹; ¹H NMR (CDCl₃) δ8.29-7.00 (m, 9H), 3.92 (s, 3M; MS m/z 348 (92, M³⁰ ), 135 (100). Anal. (C₂₀H₁₂O₄S) C, H.

2-Acetyl-naphtho[2,3-b]thiophene-4,9-dione (63b) was obtained from 63a (0.18 g, 0.80 mmol) as described for 62b to afford yellow crystals: 64% yield; mp 261-262° C.; FTIR 1669 (CO), 1651 (CO), 1590 cm⁻¹; ¹H NMR (DMSO-d₆) δ8.44 (s, 1H), 8.21-7.92 (m, 4H), 2.71 (s, 3H). Anal. (C₁₄H₈O₃S) C, H.

2-Benzoyl-naphtho[2,3-b]thiophene-4,9-dione (64b) was obtained from 64a (0.35 g, 1.21 mmol) as described for 62b to afford yellow crystals: 70% yield; mp 158-159° C.; FTIR 1667 (CO), 1652 (CO), 1634 (CO), 1594 cm⁻¹; ¹H NMR (CDCl₃) δ8.30-7.53 (m, 10H). Anal. (C₁₉H₁₀O₃S) C, H.

2-(3,4-Dimethoxybenzoyl)-naphtho[2,3-b]thiophene-4,9-dione (65b) was obtained from 65a (0.57 g, 1.63 mmol) as described for 62b to afford orange-yellow needles: 48% yield; mp 212-218° C.; FTIR 1669 (CO), 1629 (CO), 1593 cm⁻¹; ¹H NMR (DMSO-d₆) δ8.19-7.18 (m, 8H), 3.91 (s, 3H), 3.86 (s, 3H). Anal. (C₂₁H₁₄O₅S) C, H.

2-(4-Nitrobenzoyl)-naphtho[2,3-b]thiophene-4,9-dione (66b) was obtained from 66a (0.30 g, 0.90 mmol) as described for 62b to afford lemon needles: 76% yield; mp 270° C.; FTIR 1671 (CO), 1640 (CO) cm⁻¹; ¹H NMR (CDCl₃) δ8.46-7.81 (m, 9H). Anal. (C₁₉H₉NO₅S) C, H.

General Procedure for the Cleavage of Methyl Ethers. 2-(4-Hydroxybenzoyl)-naphtho[2,3-b]thiophene-4,9-dione (62c). To a solution of 62b (0.25 g, 0.72 mmol) in dry CH₂Cl₂ (50 mL) was added BBr₃ (0.70 mL, 7.18 mmol) at room temperature under N₂, and the solution was stirred at room temperature for 120 h. Then 2 N HCl (100 mL) was added, the organic layer was extracted with 2 N NaOH (3×100 mL), the combined aqueous layer was acidified with conc. HCl, and the product was dissolved in ethyl acetate. The organic layer was washed with a saturated solution of NaCl, then concentrated, and the product was crystallized at −18° C. to afford yellow-green crystals: 46% yield; mp 264-265° C.; FTIR 3553 (OH), 1669 (CO), 1648 (CO) cm⁻¹; ¹H NMR (DMSO-d₆) δ10.66 (s, 1H, exchangeable), 8.19-6.96 (m, 9H). Anal. (C₁₉H₁₀O₄S) C, H.

2-(3,4-Dihydroxybenzoyl)-naphtho[2,3-b]thiophene-4,9-dione (65c) was obtained from 65b (0.20 g, 0.53 mmol) as described for 62c. Recrystallization from toluenelethyl acetate afforded orange-yellow crystals: 65% yield; mp 272-274° C. dec; FTIR 3448 (OH), 3309 (OH), 1671 (CO), 1632 (CO) cm⁻¹; ¹H NMR (DMSO-d₆) δ10.0-9.0 (s, 2H, exchangeable), 8.20-6.93 (m, 8H). Anal. (C₁₉H₁₀O₅S) C, H.

General Procedure for the Preparation of (R,S)-Naphtho[2,3-b]furan-2-yl-alkanols. (R,S)-Naphtho[2,3-b]furan-2-yl-ethanol (73). To a solution of naphtho[2,3-b]furan (0.50 g, 2.96 mmol) in absolute Et₂O (50 mL) and tetramethylethylenediamine (0.15 mL, 1.5 mmol) was added sec-butyllithium (5.50 mL of a 1.3 M solution in hexane, 7.15 mmol) at −78° C. under N₂, and the solution was stirred at −78° C. for 4 h. Dry acetaldehyde (0.13 mL, 3.84 mmol), freshly distilled, was added at −78° C., and the solution was allowed to warm to room temperature within 12 h. Then it was treated with a half-saturated solution of NH₄Cl (250 mL), the organic layer was washed with water (250 mL), dried over Na₂SO₄, and evaporated. The residue was purified by chromatography and recrystallized from CH₂Cl₂/hexane to afford white needles: 65% yield; mp 165-167° C.; FTIR 3313 (OH) cm⁻¹; ¹H NMR (CDCl₃) δ7.99-7.19 (m, 6H), 6.72 (s, 1H), 5.6 (qu, 1H, ³J=6.6 Hz), 2.09 (s, 1H, exchangeable), 1.68 (d, 3H, ³J=6.6 Hz). Anal. (C₁₄H₁₂O₂) C, H.

(R,S)-Naphtho[2,3-b]furan-2-yl-phenylmethanol (74) was obtained from naphtho[2,3-b]furan (0.32 g, 1.89 mmol) and benzaldehyde (0.25 mL, 2.46 mmol) as described for 73. Recrystallization from hexane afforded white crystals: 42% yield; mp 100-101° C.; FTIR 3382 (OH) cm⁻¹; ¹H NMR (CDCl₃) δ7.96-7.32 (m, 11H), 6.65 (s, 1H), 5.98 (d, 1H, ³J=4.09 Hz), 2.58 (d, 1H, ³J=4.09 Hz, exchangeable). Anal. (C₁₉H₁₄O₂) C, H.

General Procedure for the Preparation of Naphtho[2,3-b]furan-2-yl-alkanones. Naphtho[2,3-b]furan-2-yl-ethanone (73a) was obtained from 73 (0.20 g, 0.94 mmol) as described for 62a, but it was stirred for 12 h to afford greenish needles: 81% yield; mp 193° C. dec (lit (Garuti et al. Farmaco Ed. Sci. 38: 527-532, 1983) 180° C.); FTIR 1679 (CO), 1632 cm⁻¹; ¹H NMR (CDCl₃) δ8.23-7.42 (m, 711), 2.67 (s, 311). Anal. (C₁₄H₁₀O₂) C, H. Naphtho[2,3-b]furan-2-yl-phenylmethanone (74a) was obtained from 74 (0.18 g, 0.66 mmol) as described for 62. Recrystallization from hexane afforded yellow needles: 86% yield; mp 140-142° C. (lit (Sen and Saxena, J. Indian Chem. Soc. 36: 283-284, 1959) 101° C.); FTIR 1634 (CO) cm⁻¹; ¹H NMR (CDCl₃) δ8.25-7.43 (m, 12H). Anal. (C₁₉H₁₂O₂) C, H.

(3,4-Dimethoxyphenyl)-naphtho[2,3-b]furan-2-yl-methanone (75a) was obtained from naphtho[2,3-b]furan (0.20 g, 1.18 mmol) and 3,4-dimethoxybenzaldehyde (0.26 g, 1.54 mmol) as described for 73, and the crude product was oxidized as described for 62a. Recrystallization from hexane afforded light-yellow crystals: 61% yield; mp 155-157° C.; FTIR 1644 (CO) cm⁻¹; ¹H NMR (CDCl₃) δ8.25 (s, 1H), 8.04 (s, 1H), 7.80-6.99 (m, 8H), 4.01 (s, 3H), 4.00 (s, 3H). Anal. (C₂₁H₁₆O₄) C, H.

General Procedure for the Preparafion of 2-Acyl-naphtho[2,3-b]furan-4,9-diones. 2-Acetyl-naphtho[2,3-b]furan4,9-dione (6) was obtained from 73a (0.11 g, 0.80 mmol) as described for 62b. Recrystallization from CH₂Cl₂/hexane afforded yellow crystals: 48% yield; mp 229-230° C. (lit (Lopez et al. J. Heterocycl Client. 21: 621-622, 1984) 222-224° C.); FTIR 1692 (CO), 1674 (CO), 1582 cm⁻¹; ¹H NMR (CDCl₃) δ8.29-7.92 (m, 4H1), 7.61 (s, 1H), 2.67 (s, 3H). Anal. (C₁₄H₈O₄) C, H.

2-Benzoyl-naphtho[2,3-b]furan-4,9-dione (74b) was obtained from 74a (0.05 g, 0.18 mmol) as described for 62b to afford yellow needles: 44% yield; mp 198-200° C.; FTIR 1674 (CO), 1659 (CO) cm⁻¹; ¹H NMR (CDCl₃) δ8.30-7.54 (m, 10H). Anal. (C₁₉H₁₀O₄) C, H.

2-(3,4-Dimethoxybenzoyl)-naphtho[2,3-b]furan:4,9-dione (75b) was obtained from 75a (0.16g, 0.48 mmol) as described for 62b. Recrystallization from CH₂Cl₂/hexane afforded lemon crystals: 40% yield; mp 242-243° C.; FTIR 1676 (CO), 1638 (CO) cm⁻¹; ¹H NMR (CDCl₃) δ8.31-7.00 (m, 8H), 4.01 (s, 3H), 4.00 (s, 3H). Anal. (C₂₁H₁₄O₆) C, H.

Various compounds of formulae I and II are shown in Tables 1 and 2, below.

TABLE 1

| | |
|---|---|
| | I |
| 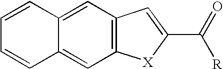 | |

| Compound | X | R |
|---|---|---|
| 73a | O | Me |
| 74a | O | Ph |
| 75a | O | 3,4-(OMe)₂-Ph |
| 63a | S | Me |
| 64a | S | Ph |
| 62a | S | 4-OMe-Ph |
| 65a | S | 3,4-(OMe)₂-Ph |
| 66a | S | 4-NO₂-Ph |

TABLE 2

| | |
|---|---|
| | II |
| 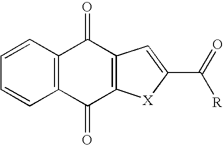 | |

| Compound | X | R |
|---|---|---|
| 6 | O | Me |
| 74b | O | Ph |
| 75b | O | 3,4-(OMe)₂-Ph |
| 63b | S | Me |
| 64b | S | Ph |
| 62b | S | 4-OMe-Ph |
| 65b | S | 3,4-(OMe)₂-Ph |
| 62c | S | 4-OH-Ph |
| 65c | S | 3,4-(OH)₂-Ph |
| 66b | S | 4-NO₂-Ph |

### Example 3

### Analogs of Formula III

The process used for the preparation of most preferred compounds of Formula III is shown in Scheme 2. An aluminum chloride catalyzed Friedel-Crafts acylation of thiophene with 3-hydroxyphthalic anhydride in methylene chloride afforded exclusively 2-hydroxy-6-(2-thenoyl)-benzoic acid (77), which was formed by the reaction of the non-hydrogen-bonded carbonyl group with thiophene. Structural proof of 77 was given by reduction with zinc in aqueous ammonia to 2-hydroxy-6-(2-thenyl)-benzoic acid (78), which in turn was converted to the corresponding methyl 2-methoxy-6-(2-thenyl)-benzoate and identified by its NOESY spectrum. Ring closure of 78 with zinc chloride in glacial acetic acid and acetic anhydride to naphtho[2,3-b]thiophene 80 proceeded with concomitant acetylation of the oxygen functions. On oxidation with chromium trioxide it afforded the quinone 81, and hydrolysis of the acetoxy function with sodium hydroxide gave the phenolic analogue 82. 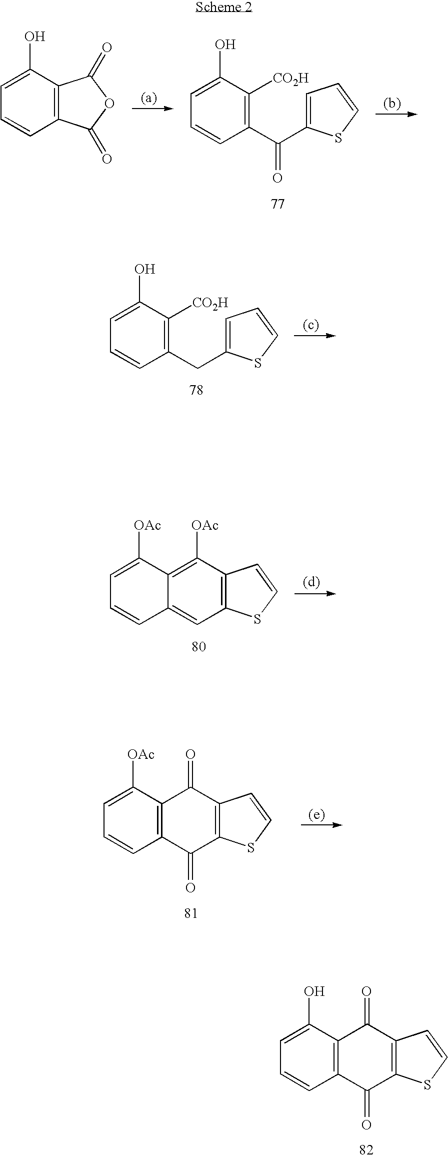
Reagents: (a) thiophene, AlCl₃, CH₂Cl₂; (c) Zn, NH₃, Δ; (c) Ac₂O, HOAc, ZnCl₂, Δ; (d) CrO₃, HOAc; (e) 6 N NaOH, Δ.

### Compounds of Scheme 2

2-Hydroxy-6-(2-thenoyl)-benzoic acid (77). To a suspension of 3-hydroxyphthalic anhydride (1.00 g, 6.09 mmol) and AICl₃ (2.44 g, 18.27 mmol) in absolute CH₂Cl₂ (20 mL) and tetramethylethylenediamine (0.15 mL, 1.5 mmol) a solution of thiophene (0.49 mL, 6.10 mmol) in absolute CH₂Cl₂ (10 mL) was added dropwise over 30 min such that the temperature of the reaction remained below 30° C. The solution was stirred at room temperature for an additional 12 h. Then it was treated with ice-water (250 mL), and the product was extracted with CH₂Cl₂ (5×100 mL). Charcoal was added to the combined organic layer, the mixture was filtered, extracted with 2 N NaOH (3×50 mL) and then acidified with conc. HCl to afford white crystals: 58% yield; mp 168-170° C.; FTIR 3432 (OH), 3151, 1681 (CO₂H), 1630 (CO) cm⁻¹; ¹H NMR (DMSO-d₆) δ11.45 (s, br, 1H, exchangeable), 8.40 (dd, 1H, ³J=4.95 Hz, ⁴J=1.21 Hz), 7.52 (dd, 1H, ³J=8.35 Hz, ³J=7.43 Hz), 7.39 (dd, 1H, ³J=3.79 Hz, ⁴J=1.21 Hz), 7.21 (dd, 1H, ³J=4.95 Hz, ³J=3.79 Hz), 7.13 (dd, 1H, ³J=8.35 Hz, ⁴J=1.09 Hz), 6.97 (dd, 1H, ³J =7.43 Hz, ⁴J =1.09 Hz). Anal. (C₁₂H₈O₄S) C,H.

2-Hydroxy-6-(2-thenyl)-benzoic acid (78). To a mixture of zinc dust (3.55 g, 54.3 mmol) and CuSO₄·5 H₂O (0.10 g) in conc. aqueous NH₃ (250 mL) was added 77 (1.39 mL 5.60 mmol). The reaction mixture was heated to reflux for 24 h, then filtered while hot, cooled to room temperature, acidified with conc. HCl, and crystallization was completed overnight in an ice-bath to afford white needles: 73% yield; mp 155-158° C.; FTIR 3427 (OH), 3290-2620 (CO₂H), 1654 (CO₂H) cm⁻¹; ¹H NMR (DMSO-d₆) δ11.70 (s, br, 1H, exchangeable), 10.50 (s, br, exchangeable), 7.29 (dd, 1H, ³J=5.13 Hz, ⁴J=1.27 Hz), 7.247.18 (m, 1H), 6.89 (dd, 1H, ³J=5.13 Hz, ³J=3.42Hz), 6.816.71 (m, 3H), 4.22 (s, 2H). Anal. (C₁₂H₁₀O₃S) C, H.

4,5-Diacetoxy-naphtho[2,3-b]thiophene (80). A mixture of 78 (0.50 g, 2.13 mmol), acetic anhydride (5 mL), glacial acetic acid (12.5 mL), and anhydrous ZnCl₂ (0.20 g, 2.13 mmol) was heated to reflux for 2 h. Then the reaction was cooled to room temperature and treated with water (100 mL). The product was filtered by suction, dissolved in CH₂Cl₂, purified by chromatography and recrystallized from CH₂Cl₂/hexane to afford pale yellow needles: 33% yield; mp 208-209° C.; FTIR 1759 (ester) cm⁻¹; ¹H NMR (CDCl₃) δ8.32 (s, 1H), 7.86-7.11 (m, 5H), 2.50 (s, 3H), 2.44 (s, 3H). Anal. (C₁₆H₁₂O₄S) C, H.

5-Acetoxy-naphtho[2,3-b]thiophene-4,9-dione (81) was obtained from 80 (0.10 g, 0.33 mmol) as described for 62b. In addition, the mother liquor was extraced with CH₂Cl₂ (30 mL), the organic layer washed with water (3×50 mL), the product was purified by chromatography using CH₂Cl₂/hexane (3/1) and recrystallized from CH₂Cl₂hexane to afford bright-yellow needles: 55% yield; mp 210° C.; FTIR 1752 (ester), 1666 (CO), 1590 cm⁻¹; ¹H NMR (CDCl₃) δ8.23 (dd, 1H, ³J=7.75 Hz, ⁴J=1.31 Hz), 7.76 (dd, 1H, ³J=8.08 Hz, ³J=7.75 Hz), 7.73 (d, 1H, ³J=5.08 Hz), 7.64 (d, 1H, ³J=5.08 Hz), 7.39 (dd, 1H, ³J=8.08 Hz, ⁴J=1.31 Hz), 2.49 (s, 3H). Anal. (C₁₄H₈O₄S) C, H.

5-Hydroxy-naphtho[2,3-b]thiophene-4,9-dione (82). A solution of 81 (0.03 g, 0.11 mmol) in CH₂Cl₂ (10 mL) and 6 N NaOH was heated to reflux for 12 h, until the yellow solution turned into deep violet. Then the reaction mixture was poured into ice-water (50 mL), acidified with conc. HCl, and the aqueous layer was extracted with CH₂Cl₂ (20 mL). The combined organic layer was dried over Na₂SO₄, purified by chromatography and recrystallized from hexane to afford orange crystals: 92% yield; mp 199-200° C.; FTIR 3440 (OH), 1656 (CO), 1632 (CO . . . HO) cm⁻¹; ¹H NMR (CDCl₃) δ12.36 (d, 1H,⁵J=0.44 Hz), 7.79 (dd, 1H, ³J=7.47 Hz, ⁴J=1.20 Hz), 7.76 (d, 1H, ³J=5.08 Hz), 7.70 (d, 1H, ³J=5.08 Hz), 7.63 (m, 1H, ³J=7.47 Hz, ³J=8.42 Hz, ⁵J=0.44 Hz), 7.29 (dd, 1H, ³J=8.42 Hz, ⁴J=1.21 Hz). Anal. (C₁₂H₆O₃S) C, H.

Various compounds of formula III are shown in Tables 3 and 4, below.

TABLE 3

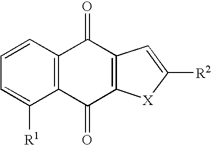

| Compound | X | R¹ | R² |
|---|---|---|---|
| 7 | O | OH | COMe |

TABLE 4

| | |
|---|---|
| | III |
| 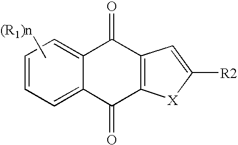 | |

| Compound | X | R¹ | R² |
|---|---|---|---|
| 45 | S | H | H |
| 81 | S | 5-OCOMe | H |
| 82 | S | 5-OH | H |

### Example 4.

### Activity of Synthetic Lapcho Analogs

Compounds of the present invention have demonstrated potent antiproliferative activity against the nontransformed human keratinocyte line HaCaT, as demonstrated by reduction in cell number over time as compared to control plates. Anthralin, an antipsoriatic drug, was used as a positive control. Antiproliferative activity was measured directly by counting the dispersed cells under a phase-contrast microscope.

HaCaT keratinocyte proliferation assay and LDH release were described previously in full detail (Muller et al. J. Med. Chem. 39: 3132-3138, 1996; Müller et al. J. Med. Chem. 37: 1660-1669, 1994). For the cancer cell line studies, exponentially growing cells were seeded at 1,000 cells per well in six-well plates and allowed to attach for 24 h. Compounds of the invention or β-lapachone, solubflized in DMSO, were added to the wells in micromolar concentrations. Control wells were treated with equivalent volumes of DMSO. After 4 h the supernatant was removed and fresh medium was added. Cultures were observed daily for 10-15 days and then were fixed and stained. Colonies of greater than 30 cells were scored as survivors.

Table 5 shows the concentrations of the compounds required to inhibit 50% of cell growth (IC₅₀). The cytotoxicity of naphthoquinones has been thought to result, at least in part, from reactive oxygen species, generated during redox cycling between the quinine and reduction products (Munday, R., Free Radic. Biol. Med., 22, 689-695, 1997), which cause peroxidative damage to membrane lipids. To assess the correlation of keratinocyte growth inhibition with membrane damage, the release of lactate dehydrogenase from the treated cells was also quantitated.

TABLE 5

| | | | | | AA^{a} in | | AA^{c} in Cancer Cell Lines | | |
|---|---|---|---|---|---|---|---|---|---|
| | | | | | HaCaT Cells | LDH^{b} | IC₅₀ (μM) | | |
| Compound | X | R | R1 | R2 | IC₅₀ (μM) | (mU) | DLD1 | SW480 | MCF7 |

Compounds of Formula I

| | | | | | | | | | |
|---|---|---|---|---|---|---|---|---|---|
| 73a | O | Me | NA | NA | >5 | ND | >32 | >32 | >32 |
| 74a | O | Ph | NA | NA | 1.9 | 142 | 7.6 | 12 | ≦1 |
| 75a | O | 3,4-(OMe)₂—Ph | NA | NA | >5 | ND | 14 | 30 | 12 |
| 63a | S | Me | NA | NA | 5.0 | ND | >32 | >32 | >32 |
| 64a | S | Ph | NA | NA | 0.3 | 122 | 1.5 | ≦1 | ≦1 |
| 62a | S | 4-OMe—Ph | NA | NA | >5 | ND | >32 | >32 | >32 |
| 65a | S | 3,4-(Ome)₂—Ph | NA | NA | >5 | ND | >32 | >32 | >32 |
| 66a | S | 4-NO₂—Ph | NA | NA | >5 | ND | >32 | >32 | >32 |

Compounds of Formula II

| | | | | | | | | | |
|---|---|---|---|---|---|---|---|---|---|
| 6 | O | Me | NA | NA | 0.5 | 331 | 0.8 | ≦1 | ≦1 |
| 74b | O | Ph | NA | NA | 0.7 | 222 | ND | ND | ND |
| 75b | O | 3,4-(OMe)₂—Ph | NA | NA | 2.5 | 250 | 1.4 | ND | ND |
| 63b | S | Me | NA | NA | 0.3 | 134 | 1 | 4 | ≦1 |
| 64b | S | Ph | NA | NA | 1.7 | ND | 1.3 | 4 | ≦1 |
| 62b | S | 4-OMe—Ph | NA | NA | >5 | ND | 5.5 | 16 | 3 |
| 65b | S | 3,4-(OMe)₂—Ph | NA | NA | 0.8 | 137 | 2.6 | 10 | ≦1 |
| 62c | S | 4-OH—Ph | NA | NA | 2.7 | 123 | 5.3 | 12 | 2 |
| 65c | S | 3,4-(OH)₂—Ph | NA | NA | 1.5 | 118 | 11 | 20 | 8 |
| 66b | S | 4-NO₂—Ph | NA | NA | 4.0 | ND | 25 | ND | ND |

Compounds of Formula III

| | | | | | | | | | |
|---|---|---|---|---|---|---|---|---|---|
| 7 | O | NA | 8-OH | COMe | 0.3 | 346 | ND | ND | ND |
| 45 | S | NA | H | H | >5 | 222 | ND | ND | ND |
| 81 | S | NA | 5-OCOMe | H | 1.4 | 160 | ND | ND | ND |
| 82 | S | NA | 5-OH | H | 1.0 | 117 | ND | ND | ND |
| α-lapachone | NA | NA | NA | NA | 10 | ND | ND | ND | ND |
| β-lapachone | NA | NA | NA | NA | 0.7 | 329 | 4 | 4 | ≦1 |
| anthralin | NA | NA | NA | NA | 0.7 | 294 | NA | NA | NA |
| vehicle | NA | NA | NA | NA | NA | 135 | NA | NA | NA |

^{a}Antiproliferative activity against HaCaT cells. Inhibition of cell growth was significantly different with respect to that of the control, N = 3, p < 0.05.
^{b}Activity of LDH (mU) release in HaCaT cells after treatment with 2 μM test compound, N = 3, SD < 10%, p < 0.05. NA = not applicable.
^{c}Antiproliferative activity against colon cancer cell lines DLD1 and SW480 and breast cancer cell line MCF7. ND = not determined. NA = not applicable.

As shown in Table 5, treatment of HaCaT cells with anthralin was effective at inhibition of proliferation (IC₅₀=0.7 μM) but caused substantial cellular damage, with LDH release significantly higher than vehicle controls. Similarly, β-lapachone and the 2-acetylated naphtho[2,3-b]furan4,9-diones (compounds 6 and 7) inhibited cell proliferation but caused significant LDH release as compared to vehicle. However, several of the thiophene analogs (compounds 63a, 64b, 65b, 65c, 81 and 82) inhibited cell proliferation at concentrations comparable to β-lapachone and the furan analogs but without significant elevation of LDH release over the vehicle control.

Compounds of this invention were also effective at inhibiting proliferation of human cancer cells including cells from the colon cancer lines DLD1 and SW480 and from the breast cancer line MCF7. As shown in Table 5, IC₅₀ values in the low micromolar range and below were obtained for several of these compounds in all three cancer cell lines

The antiproliferative activity of the present synthetic lapacho derivative compounds suggests that compounds of the invention may be expected to show wide anticancer activity. For example, the compounds of the invention may be effective for treating cancers such as breast cancer, leukemia, lung cancer, ovarian cancer, brain cancer, liver cancer, pancreatic cancer, prostate cancer, and colorectal cancer. These treatments may be accomplished utilizing the present lapacho derivative compounds (Formula I, II or III) alone or in combination with other chemotherapy agents or with radiation therapy. In a preferred embodiment the present lapacho derivative compounds are used for the prevention or treatment of cancer (e.g., as a preventative drug) by preventing cancer cell formation.

As described in part above, a variety of cancer cell lines could be used to determine the effectiveness of the novel lapacho derivatives of the present invention, including SK-OV-3 and OVCAR-3 human ovarian carcinoma cells; SW480, HT-29 and HCT-116 human colon carcinoma cells; MCF-7 and MDA-MB-231 human breast carcinoma cells; MIA PACA-2 and BXPC-3 human pancreatic carcinoma cells; NCI-H226 and A549 human lung carcinoma cells; and DU-145 and PC-3 human prostate cancer cells. Since β-lapachone induces apoptosis only in cancer cell lines and not in normal cells (Li., Y, et al., PNAS, (2003), 100(5), 2674-2678) the present compounds can also be tested in a panel of normal cell lines including NCM 460 normal colonic epithelial cells and MCF 10A normal breast epithelial cells.

The results of experiments with β-lapachone and similar chemical compounds have shown that the present lapacho derivatives may have a strong apoptotic effect on a variety of human cancer cells and that they can inhibit growth of other human cancer cells.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A compound of formula I: 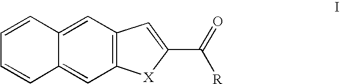
wherein X is O or S; and R is straight-chained or branched C₁-C₆ alkyl, aryl, substituted aryl, or straight-chained or branched alkylaryl, or a pharmaceutically acceptable salt thereof; wherein 1) R is not methyl; 2) where X is O, R is not bromomethyl, unsubstituted phenyl, or phenyl substituted at the 4-position with methyl, chloro, ethenyl, or 2′-chloroethyl; and 3) where X is S, R is not 2-carboxyphenyl.

2. A compound according to claim 1, wherein X is S, and R is aryl or substituted aryl.

3. A compound according to claim 1 or 2, wherein said substituted aryl is substituted with at least one substituent selected from the group consisting of hydroxyl, alkoxy, C₁-C₆ alkyl, nitro, halogen, carboxyl and carboxyalkyl.

4. A compound according to claim 1, wherein X is S, and R is phenyl.

5. A compound of formula II: 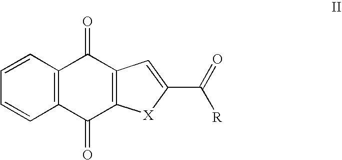
wherein X is O or S; and R is straight-chained or branched C₁-C₆ alkyl, aryl, substituted aryl, or straight-chained or branched alkylaryl, or a pharmaceutically acceptable salt thereof; wherein R is not methyl.

6. A compound according to claim 5, wherein X is O or S, and R is C₁-C₆ alkyl, aryl or mono- or di-substituted aryl.

7. A compound according to claim 5 or 6, wherein said substituted aryl is substituted with at least one substituent selected from the group consisting of hydroxyl, alkoxy, alkyl, nitro, halogen, carboxyl and carboxyalkyl.

8. A compound according to claim 5, wherein X is O, and R is phenyl.

9. A compound according to claim 5, wherein X is O, and R is 3,4-dimethoxyphenyl.

10. A compound according to claim 5, wherein X is S, and R is phenyl.

11. A compound according to claim 5, wherein X is S, and R is 3,4-dimethoxyphenyl.

12. A compound according to claim 5, wherein X is S, and R is 4-hydroxyphenyl.

13. A compound according to claim 5, wherein X is S, and R is 3,4-dihydroxyphenyl.

14. A compound of formula III: 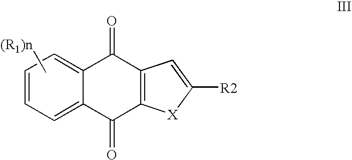
wherein X is O or S; R₁ is independently at each incidence hydroxyl, alkoxyl, C₁-C₆ alkyl, nitro, halogen, carboxyl or carboxyalkyl; R₂ is hydrogen or —C(O)—R₃, R₃ is straight-chained or branched C₁-C₆ alkyl, aryl, substituted aryl, or straight-chained or branched alkylaryl; and n is 0, 1 or 2; or a pharmaceutically acceptable salt thereof; wherein 1) where X is O, R₂ is not H; 2) where X is O, and R₂ is —C(O)—R₃, and R₃ is methyl, then R₁ is not hydroxyl or methoxy; and 3) where X is S and R₂ is H, then n is 1 and R₁ is selected from —OH and —OC(O)-alkyl(C₁-C₆); and 4) where X is S and R₂ is —C(O)—R₃, and R₃ is methyl, then R₁ does not represent a 7-acetyl group.

15. A compound according to claim 14, wherein X is S, R₁ is hydroxyl or alkylcarbonyl, R₂ is hydrogen, and n is 1.

16. A compound according to claim 14, wherein X is S, R₁ is 5-carboxymethyl, R₂ is hydrogen, and n is 1.

17. A compound according to claim 14, wherein X is S, R₁ is 5-hydroxyl, R₂ is hydrogen, and n is 1.

18. A pharmaceutical composition comprising a therapeutically effective amount of a compound according to claim 1 in combination with a pharmaceutically acceptable carrier.

19. A pharmaceutical composition comprising a therapeutically effective amount of a compound according to claim 4 in combination with a pharmaceutically acceptable carrier.

20. A pharmaceutical composition comprising a therapeutically effective amount of a compound according to claim 5 in combination with a pharmaceutically acceptable carrier.

21. A pharmaceutical composition comprising a therapeutically effective amount of a compound according to any one of claims 8-13 in combination with a pharmaceutically acceptable carrier.

22. A pharmaceutical composition comprising a therapeutically effective amount of a compound according to claim 14 in combination with a pharmaceutically acceptable carrier.

23. A pharmaceutical composition comprising a therapeutically effective amount of a compound according to claims 16 or 17 in combination with a pharmaceutically acceptable carrier.

24. A method of treating or preventing cell proliferative disorders comprising administering to a mammal a therapeutically effective amount of a compound according to any of claims 1-17.

25. The method according to claim 24, wherein the compound is administered as a pharmaceutical composition of claim 18.

26. The method according to claim 24, wherein the compound is administered as a pharmaceutical composition of claim 19.

27. The method according to claim 24, wherein the compound is administered as a pharmaceutical composition of claim 20.

28. The method according to claim 24, wherein the compound is administered as a pharmaceutical composition of claim 21.

29. The method according to claim 24, wherein the compound is administered as a pharmaceutical composition of claim 22.

30. The method according to claim 24, wherein the compound is administered as a pharmaceutical composition of claim 23

31. A method of treating cancer or precancerous conditions or preventing cancer comprising administering to a mammal in need thereof a therapeutically effective amount of a pharmaceutical composition according to claim 18.

32. A method of treating cancer or precancerous conditions or preventing cancer comprising administering to a mammal in need thereof a therapeutically effective amount of a pharmaceutical composition according to claim 19.

33. A method of treating cancer or precancerous conditions or preventing cancer comprising administering to a mammal in need thereof a therapeutically effective amount of a pharmaceutical composition according to claim 20.

34. A method of treating cancer or precancerous conditions or preventing cancer comprising administering to a mammal in need thereof a therapeutically effective amount of a pharmaceutical composition according to claim 21.

35. A method of treating cancer or precancerous conditions or preventing cancer comprising administering to a mammal in need thereof a therapeutically effective amount of a pharmaceutical composition according to claim 22.

36. A method of treating cancer or precancerous conditions or preventing cancer comprising administering to a mammal in need thereof a therapeutically effective amount of a pharmaceutical composition according to claim 23.

37. A method of treating or preventing psoriasis comprising administering to a mammal in need thereof a therapeutically effective amount of a pharmaceutical composition according to claim 18.

38. A method of treating or preventing psoriasis comprising administering to a mammal in need thereof a therapeutically effective amount of a pharmaceutical composition according to claim 19.

39. A method of treating or preventing psoriasis comprising administering to a mammal in need thereof a therapeutically effective amount of a pharmaceutical composition according to claim 20.

40. A method of treating or preventing psoriasis comprising administering to a mammal in need thereof a therapeutically effective amount of a pharmaceutical composition according to claim 21.

41. A method of treating or preventing psoriasis comprising administering to a mammal in need thereof a therapeutically effective amount of a pharmaceutical composition according to claim 22.

42. A method of treating or preventing psoriasis comprising administering to a mammal in need thereof a therapeutically effective amount of a pharmaceutical composition according to claim 23.
